(19) United States
(12) Patent Application Publication
(10) Pub. No.: US 2021/0369610 A1
(43) Pub. Date: Dec. 2, 2021

(54) LIQUID GRIP LOTION

(71) Applicant: Better Grip Sports Products, Inc., Sioux Falls, SD (US)

(72) Inventor: Elise M. Thullen, Sioux Falls, SD (US)

(73) Assignee: Better Grip Sports Products, Inc., Sioux Falls, SD (US)

(21) Appl. No.: 17/334,537

(22) Filed: May 28, 2021

Related U.S. Application Data
(60) Provisional application No. 63/031,472, filed on May 28, 2020.

(51) Int. Cl. A61K 9/06; A61K 31/167; A61K 47/02; A61K 47/10; A61K 47/34; A61K 9/00

(52) U.S. Cl. CPC A61K 9/06; A61K 31/167; A61K 9/0014; A61K 47/10; A61K 47/34; A61K 47/02

(57) ABSTRACT

A topical liquid grip lotion is disclosed in multiple embodiments. The topical liquid grip lotion includes embodiments which are longer lasting that prior products and also includes embodiments which are formulated to provide relief from temporary user discomfort or pain that may associated with the underlying activity by the user in areas where the topical lotion is applied. The certain embodiments of the liquid sports grip aid/liquid hand chalk include about 1% lidocaine. In addition to increasing the amount of time of use per application, these embodiments provide a pain reducing agent which can be exceptionally useful for athletes and individuals who experience soreness in their hands, feet, and body when training. Alternative embodiments include formulations are substantially non-irritating due to the topical use of the product, and not intended for ingestion. It is unique that as not only do the embodiments immediately dry hands, feet, and body but can reduce soreness.

### CROSS-REFERENCE TO RELATED APPLICATIONS

This application claims priority to U.S. Provisional Patent Application No. 63/031,472, filed May 28, 2020 and entitled “LIQUID GRIP LOTION,” which is incorporated by reference herein in its entirety as if fully set forth herein.

### BACKGROUND OF THE INVENTION

Excess perspiration or sweating of the hands and feet is an issue that afflicts many people. The presence of such perspiration can be detrimental when the person is engaged in a sporting activity or other activity where dry hands or feet is important. For example, in golf, perspiration on a person's hands can cause mistakes or errors due to a slipped grip resulting from excessive perspiration on the hand(s). Likewise, excessive perspiration on the hands or feet of a gymnast can cause slips or falls that detrimentally impact the performance being attempted.

A number of products are commercially and widely available which are designed to help prevent sweat and perspiration by individuals who experience sweat and perspiration in their hands and feet. While these products achieve varying degrees of success as moisture and sweat repellants, research continues in an effort to develop more effective ways to keep hands dry.

Sweat and perspiration in the hands and feet is typically caused by two factors. First, elevated body temperature can be a factor resulting in hands and feet perspiring. The second, and more common reason, is that stress and anxiety can cause hands and feet to perspire at a undesirable level and with no apparent cause other than nervousness and stress. Thus, for example, the more a person experiences a stressful environment, or feels nervous or anxious, the more likely the person is to have sweaty hands. This is very common in sports and activities that involve learning and mastering new physical skills. Such sports can include, but are not limited to, developing a better game of golf, rock climbing or having a firmer grip on a tennis racket. Furthermore, the more developed an athlete is, the more he or she may train. This can lead to longer sessions of the activity in which they seek to improve or excel, and which may additionally lead to pain and soreness in the hands and feet.

By using a product that helps aid in the prevention of moisture and perspiration, the individual can have a more productive training session, and also possibly train longer. While there are prior know products available that can help with keeping hands and feet dry, they need constant reapplication to continue producing this effect, which is undesirable to the individual as they constantly interrupt their training to reapply the prior products.

One or more embodiments of the present disclosure can reduce or prevent the accumulation of excess moisture and perspiration of the hands and feet. One or more embodiments of the present disclosure product can last longer than conventional products. A product which can last longer without as many reapplications, permits more time for training without interruption.

Likewise one or more embodiments of the current disclosure can repel water as well as sweat, and not only is able to be applied to hands and feet, but other areas of the body topically. Additionally, one or more embodiments of the present disclosure facilitates the use of hand chalk without the messiness that traditional chalk produces. Such embodiments can be an excellent way to apply hand chalk in a liquid form which can facilitate easier application and use with substantially less mess or lingering particulates.

Additionally, one or more formulations of the product described herein can also provide comfort or aid regarding the soreness that can arise in the hands and feet of a user. These formulations can allow the user to maintain or exceed their current endurance levels through training in their desired activity or sport.

Lastly, one or more embodiments of the present disclosure can be cleaned from the application area by simply by washing the area with soap and water. This too supports the simplicity of use with less mess or residual undesired matter.

### SUMMARY OF THE DISCLOSURE

The present disclosure relates to one or more novel embodiments of a topically applied liquid grip lotion product which can enhance the ability of the user to reduce excessive moisture in areas of the user's body where it is applied. This includes areas such as the hands or feet of the user.

One or more formulations of the present disclosure can act as a liquid sports grip aid or a liquid hand chalk.

One or more of the disclosed embodiments can last longer than prior products.

Additionally, one or more of the disclosed embodiments can ease or decrease pain or soreness in the hands, feet, and/or body area where applied.

One or more embodiments of the present disclosure can alternatively make the user's participation in the activity more enjoyable or successful during and after the activity.

These advantages, and the advantages known to a person of skill in the art from a review of this disclosure, can facilitate longer, more productive and/or more enjoyable training or performance of the underlying activity both during the activity and after.

### DETAILED DESCRIPTION OF THE INVENTION

The terminology employed herein to describe certain ingredients of the embodiments of the present disclosure include names of chemicals that are known and available to laboratories and holders of an Industrial Alcohol Permit through the Tax and Trade Bureau (TTB).

One or more embodiments of the present disclosure, in addition to increasing the amount of time of use per application, can also provide a reduction in pain which can be exceptionally useful for athletes and individuals who experience soreness in their hands, feet and body.

For example, one embodiment includes approximately about 1% Lidocaine Hydrochloride and provides a novel liquid sports grip aid/liquid held chalk which dramatically reduces sweat and perspiration in the hands, feet, and body; lasts longer, and decreases pain and soreness in the hands, feet, and body when applied. This results in activities and training facilitating longer, more productive workouts.

An alternative embodiment does not include Lidocaine, for use by persons who prefer a lidocaine-free version due to preferences or where the user has or may be susceptible to a lidocaine allergy.

The formulation is nonirritating due to the topical use of the product, and not intended for ingestion; rather only topically. The embodiments of the present disclosure are unique in that they facilitate an individual maintaining a hand grip that can last longer, with less pain, with less reapplication, as well as a less messy usage, than conventional products.

Preferably, one or more of the embodiments of the present disclosure use various general ingredients. The ingredients should be of a quality and purity suitable for topical use, and can include one or more of the following, and any known substitutes therefor:

### General Ingredients:

### SD Alcohol 40

### Fumed Silica

### Phenyltrimethicone

### Cyclomethicone

### Lidocaine Hydrocloride

### Polyethylene Glycol 300

The below examples include formulations of the liquid grip that are intended to be exemplary and not limiting on the scope of the disclosure of the embodiments of the present disclosure.

### Example 1

In this example formulation, the liquid grip embodiment includes the following ingredients by weight percent:

| Ingredient | Weight Percent |
|---|---|
| SD Alcohol 40 | Between about 73.5% to about 83.5%, |
| | preferably about 80.50% |
| Fumed Silica | Preferably about 10.88% |
| Phenyltrimethicone | Between about 4.13% to about 4.57%, |
| | preferably about 4.35% |
| Cyclomethicone | Between about 2.07% to about 2.29%, |
| | preferably about 2.18% |
| Lidocaine | Between about 1.04% to about 1.14%, |
| Hydrocloride | preferably about 1.09% |
| Polyethylene | Preferably about 1.00% |
| Glycol 300 | |

Each of the specific ingredients listed above are known and commercially available, and may have known substitutes, all of which are intended to be included in the list of ingredients specified in Example 1 above.

### Example 2

An alternative formulation of the liquid grip of the present disclosure was prepared having the following ingredients:

| Ingredient | Weight percent range | Embodiment |
|---|---|---|
| Ethanol | Between about 73.5% | About 78.2% |
| | to about 83.5% | |
| Phenyl | Between about 4.13% | About 4.39% |
| Trimethicone | to about 4.57% | |
| Cyclomethicone | Between about 2.07% | About 2.24% |
| | to about 2.29% | |
| Lidocaine HCl | Between about 1.04% | About 1.10% |
| | to about 1.14% | |

Each of the specific ingredients listed above are known and commercially available, and may have known substitutes, all of which are intended to be included in the list of ingredients specified in Example 1 above. In addition, by way of example, the Ethanol in this Example 2 can be SD Alcohol 40 or a known substitute.

### Example 3

An alternative formulation of the liquid grip of the present disclosure was prepared having the following ingredients:

| Ingredient | Weight percent range | Embodiment |
|---|---|---|
| Ethanol | Between about | About 78.9% |
| | 73.5% to 83.5% | |
| Phenyl | Between about | About 4.47% |
| Trimethicone | 4.13% to 4.57% | |
| Cyclomethicone | Between about | About 2.27% |
| | 2.07% to 2.29% | |

Each of the specific ingredients listed above are known and commercially available, and may have known substitutes, all of which are intended to be included in the list of ingredients specified in Example 1 above. In addition, by way of example, the Ethanol in this Example 3 can be SD Alcohol 40 or a known substitute.

### Example 4

An alternative formulation of the liquid grip of the present disclosure was prepared having the following ingredients:

| | Weight | |
|---|---|---|
| Ingredient | percent range | Embodiment |
| SD ALCOHOL 40B | See above | About 78.9% |
| FUMED SILICA | See above | About 12.48% |
| PHENYLTRIMETHICONE | See above | About 4.35% |
| CYCLOTRIMETHICONE | See above | About 2.18% |
| POLYETHYLENE | See above | About 1% |
| GLYCOL 300 | | |

Any of the Examples can include missing ingredients from another example, or water, to make up the remaining components of the liquid grip embodiment.

Results with various embodiments have been used for tennis, golf, gymnastics, basketball, weightlifting, and pole dancing, and have been well received. By way of example, customer comments have included at least the following:
“Not as tacky as <alternative product> but definitely worth buying. It makes the pole burn not sting as much when doing drops and skin grips.”
“AMAZING. I'm a beginning pole fitness dancer and this stuff is life changing! I recommend it to everyone”
“I wasn't able to dunk before until now !”
“The best product to improve grip drastically! Highly recommend and I've tried everything else on the market. We use it in our gym instead of chalk to perform Olympic lifts and on pull up bars. Also amazing if you are use a pole for fun and fitness! When I first grabbed the pole, I thought there is no way I can pull my weight off the ground! Then after using better grip I could! Love it!”
“I like it! It is comparable to <alternative product>, but I like that it helped my hands feel less sore. I bought some to try and I really like it!”
“Perfect for pole fitness classes”
“I ordered this a couple months ago, and I'm still on my first bottle. A little goes a long way. I tried this after <alternative product> started becoming more expensive, and I never pole dance or train without it. I really like how it has a less chalky residue than other grips I've tried, and will continue to use this instead of <alternative product>.”

1. A topical liquid grip lotion which provides relief from soreness of the user consisting of essentially the following ingredients:
SD Alcohol 40
Fumed Silica
Phenyl Trimethicone
Cyclomethicone
Lidocaine Hydrocloride
Polyethylene Glycol 300

2. A topical liquid grip lotion of claim 1, wherein the ingredients are present in essentially the following percentages by weight:

| Ingredient | Weight percent range |
|---|---|
| SD Alcohol 40 | Between about 73.5% to about 83.5%, |
| | preferably about 80.50% |
| Fumed Silica | Preferably about 10.88% |
| Phenyl | Between about 4.13% to about 4.57%, |
| Trimethicone | preferably about 4.35% |
| Cyclomethicone | Between about 2.07% to about 2.29%, |
| | preferably about 2.18% |
| Lidocaine | Between about 1.04% to about 1.14%, |
| Hydrocloride | preferably about 1.09% |
| Polyethylene | Preferably about 1.00% |
| Glycol 300 | |

3. A topical liquid grip lotion consisting of essentially the following active ingredients in essentially the following percentages by weight:

| Ingredient | Weight percent range | Embodiment |
|---|---|---|
| Ethanol | Between about 73.5% | About 78.2% |
| | to about 83.5% | |
| Phenyl | Between about 4.13% | About 4.39% |
| Trimethicone | to about 4.57% | |
| Cyclomethicone | Between about 2.07% | About 2.24% |
| | to about 2.29% | |

4. A topical liquid grip lotion of claim 3, wherein an additional active ingredient is added in essentially the following percentage by weight:

| Ingredient | Weight percent range | Embodiment |
|---|---|---|
| Lidocaine HCl | Between about 1.04% | About 1.10% |
| | to about 1.14% | |

5. The topical liquid grip lotion of claim 3, wherein the active ingredients are present in essentially the following percentages by weight:

| Ingredient | Weight percent range | Embodiment |
|---|---|---|
| Ethanol | Between about 73.5% to 83.5% | About 78.9% |
| Phenyl | Between about 4.13% to 4.57% | About 4.47% |
| Trimethicone | | |
| Cyclomethicone | Between about 2.07% to 2.29% | About 2.27% |

6. A topical liquid grip lotion of claim 3, wherein additional active ingredients are added or substituted and the active ingredients are present in essentially the following percentages by weight:

| Ingredient | Weight percent range |
|---|---|
| SD Alcohol 40 | Between about 73.5% to about 83.5%, |
| | preferably about 80.50% |
| Fumed Silica | Preferably about 10.88% |
| Phenyl Trimethicone | Between about 4.13% to about 4.57%, |
| | preferably about 4.35% |
| Cyclomethicone | Between about 2.07% to about 2.29%, |
| | preferably about 2.18% |
| Polyethylene | Preferably about 1.00% |
| Glycol 300 | |

7. A topical liquid grip lotion of claim 6, wherein an additional active ingredient is added in essentially the following percentage by weight:

| Ingredient | Weight percent range |
|---|---|
| Lidocaine | Between about 1.04% to about |
| Hydrocloride | 1.14%, preferably about 1.09% |

8. A topical liquid grip lotion of claim 5, wherein an additional active ingredient is added in essentially the following percentage by weight:

| Ingredient | Weight percent range | Embodiment |
|---|---|---|
| Lidocaine | Between about 1.04% | About 1.10% |
| HCl | to about 1.14% | |
